(19) United States
(12) Patent Application Publication
(10) Pub. No.: US 2020/0174451 A1
(43) Pub. Date: Jun. 4, 2020

(54) SYSTEM AND METHODS FOR PATIENT SPECIFIC THREE DIMENSIONAL MODELS WITH INTERVENING LAYERS

(71) Applicant: Mediprint, LLC, Goshen, NY (US)

(72) Inventor: Brent Chanin, Goshen, NY (US)

(73) Assignee: Mediprint, LLC, Goshen, NY (US)

(21) Appl. No.: 16/727,018

(22) Filed: Dec. 26, 2019

Related U.S. Application Data
(63) Continuation-in-part of application No. 16/515,908, filed on Jul. 18, 2019.
Application No. 16/515,908 is a continuation of application No. 16/372,947, filed on Apr. 2, 2019.
Application No. 16/515,908 is a continuation of application No. 16/373,114, filed on Apr. 2, 2019.
(60) Provisional application No. 62/651,530, filed on Apr. 2, 2018.
(60) Provisional application No. 62/651,545, filed on Apr. 2, 2018.
(60) Provisional application No. 62/651,530, filed on Apr. 2, 2018.
(60) Provisional application No. 62/651,545, filed on Apr. 2, 2018.
(60) Provisional application No. 62/651,562, filed on Apr. 2, 2018.
(60) Provisional application No. 62/699,827, filed on Jul. 18, 2018.
(60) Provisional application No. 62/788,179, filed on Jan. 4, 2019.

(51) Int. Cl. G05B 19/4099; G16H 30/20; B33Y 50/00; G06F 3/12

(52) U.S. Cl. CPC G05B 19/4099; G16H 30/20; B33Y 50/00; G06F 3/1206; G06F 3/1286; G06F 3/1232; G05B 2219/49007; G05B 2219/35134; G06F 3/1297

(57) ABSTRACT

The present disclosure describes systems and methods to create improved three-dimensional models which may be fabricated from multiple materials. In some examples, intervening layers may be used to create better composite structures of the multiple materials. In other examples, intervening layers may convey information such as the depiction of borders. In some embodiments, the models are of one or more body parts of a patient, and accordingly are built using patient-specific data and/or a database of generic body parts.

### CROSS REFERENCES TO RELATED APPLICATIONS

This application claims priority to U.S. Provisional Patent Application Ser. No. 62/788,179 entitled “SYSTEM AND METHODS FOR PATIENT SPECIFIC THREE DIMENSIONAL MODELS WITH INTERVENING LAYERS”, filed on Jan. 4, 2019 as a continuation in part to U.S. Non-Provisional patent application Ser. No. 16/515,908 entitled “SYSTEM AND METHOD FOR MECHANICAL FASTENING DESIGN”, filed on Jul. 18, 2019 which is a continuation to U.S. Non-Provisional patent application Ser. No. 16/372,947 tilted “APPARATUS AND METHODS FOR CREATION OF A PATIENT-SPECIFIC ANATOMICAL MODEL”, filed on Apr. 2, 2019 and as a continuation to U.S. Non-Provisional patent application Ser. No. 16/373,114 entitled “APPARATUS AND METHODS FOR CREATION OF A PATIENT-SPECIFIC BODY PART OR MEDICAL INSERT”, filed on Apr. 2, 2019 each of which are relied upon and incorporated herein by this reference.

### FIELD OF THE DISCLOSURE

The present disclosure relates to systems and methods to create patient-specific three-dimensional models which may be taken apart and reassembled with ease and accuracy and which incorporate layered structures for performance and demonstration purposes.

### BACKGROUND OF THE DISCLOSURE

Three-dimensional models may be realized in physical space in numerous manners. It may be desirable for the internal structure of a physically rendered model to be viewable from the exterior of the model. However, in various examples, the internal structure of the physically rendered model may become impossible or difficult to visualize due to its being imbedded behind externally displayed features. Accordingly, in some examples, the physical three-dimensional model may comprise materials that transmit light and thus allow for an internal structure to be visualized if that structure is able to be understood from the nature of the transmitted light. For example, color variation may be used to display shapes and contours of the internal structure. Even so, the visualization of the internal structure may be difficult, and distortions of the visualized structure occur. For this reason, it may be common to create cross sections of the physical model and display the structure on the surface of the cross section.

Cross-sectioned pieces may have complicated shapes, and although it is typically straightforward to separate pieces that have been cut in cross section, it may be very difficult to reorient these pieces in their correct configuration. It would be very helpful if improved model generation techniques were developed which allowed for easy separation as well as accurate and easy reassembly. Additional improvements accrue from techniques which enable nimbleness of assembly, which are cross compatible, and which can be operated with one hand. Other desirable attributes may include solutions which have joints and are articulated, solutions which are attractive, are sturdy, and which are intuitive.

A novel class of such three-dimensional models includes medical models which have many uses in the medical industry. Non-specific models of human physiology (i.e., models that are not limited to a specific person) are useful as learning tools in contexts such as anatomy class in medical school, medical instructions that affect a group of patients (e.g., for conditions such as pregnancy, insertion of an intra-uterine device, disorders such as enlarged hearts, etc.), cardio-pulmonary resuscitation (CPR) training, and so forth.

In contrast, a patient-specific medical model is a medical model of at least a portion of the anatomy (internal or external) of one specific patient, at one point in time. The patient-specific medical model may be useful to a medical practitioner to understand a patient's medical state, to plan a medical procedure for the patient, and/or to perform the medical procedure for the patient. A modality of the patient-specific medical model may refer to the technology used to obtain the patient-specific medical model.

One technology of the background art to obtain a patient-specific medical model is by computed tomography (CT) scan, also referred to as computed axial tomography or computer aided tomography (CAT) scan. CT and CAT scans make use of computer-processed combinations of many X-ray measurements taken from different angles to produce cross-sectional (tomographic) images (virtual “slices”) of specific areas of a patient or other scanned object, allowing a medical professional to see inside the patient without cutting open the patient.

Another technology of the background art to obtain a patient-specific medical model is by magnetic resonance imaging (MRI). An MRI scanner uses strong magnetic fields, electric field gradients, and radio waves to generate images of the organs in the body. The images generally are of a planar slice through the patient's body. MRI does not involve X-rays and the use of ionizing radiation, in contrast to CT or CAT scans. Compared with CT scans, MRI scans typically take longer and are louder, and they usually need the patient to enter a narrow, confining tube. In addition, patients with some medical implants or other non-removable metal inside the body may be unable to undergo an MRI examination safely.

Data from a CT scan or MRI scan is ordinarily stored in accordance with NEMA PS3/ISO 12052, Digital Imaging and Communications in Medicine (DICOM) Standard, National Electrical Manufacturers Association, Rosslyn, Va., USA (available free at http://medical.nema.org/). The DICOM standard covers both the formats to be used for storage of digital medical images and related digital data, and the protocols to be adopted to implement several communication services which are useful in the medical imaging workflow. DICOM facilitates cross-vendor interoperability among devices and information systems dealing with digital medical images.

Having an accurately and timely patient-specific medical model is extremely important to the successful performance of critical procedures such as surgeries, or treatment of delicate tissue such the eye or nerve tissue. Surgeries may include cardiac surgery, neurosurgery, oncology surgery, ophthalmologic surgery, orthopedic surgery, plastic surgery, and so forth. A preferred physical resolution (e.g., slice thickness) in the background art of the patient-specific medical model may depend upon the type of surgery and the modality used to obtain the model data, and generally ranges from less than about 2.0 mm to less than about 0.7 mm.

In connection with the critical procedures, a replacement body part may be needed, e.g., a prosthetic or a replacement for an internal body part. For example, the replacement for an internal body part may be replacement for a joint (e.g., hip, knee, elbow, etc.), a bone (e.g., a vertebrae), a cartilage (e.g., ear, nose, etc.), reconstructive surgery (e.g., to correct a birth defect, to correct a traumatic injury, etc.), a blood vessel to replace a clogged vessel, and so forth. The replacement body part may be used for other purposes, instead of or in addition to being used as a replacement, such as being used as a physical model of a body part for illustrative or learning purposes, etc.

All of these different medical models share the general needs as mentioned initially for improvements that make it easy to demonstrate internal structure and disassemble and reassemble them for various purposes. The models may be formed of varying materials with different properties and there may be numerous issues that arise from building structures with different materials. Therefore, it may be important to incorporate strategies that improve the nature of models formed from multiple materials.

### SUMMARY OF THE DISCLOSURE

Accordingly, apparatus and methods to improve sectioned physical models made of multiple materials are presented. In some examples, a first material may be fabricated into a preform, wherein the first material has a first material characteristic and the shape of the first preform is based upon a calculation performed upon one or more scans of a patient's body part. In some embodiments, one or more scans of a patient's body part may be stored in a database of an imaging scan acquired for a particular patient. A second material may be applied to at least a portion of the preform, wherein the second material may have a second material characteristic. Embodiments may include multiple layers, such as a third material may be applied upon the second material. In some examples, the second material characteristic may be that the second material improves bonding between the first material and the third material. In some other examples, the second material characteristic may be that the second material provides a clear delineation between the first material and the third material. In still further examples, the second material characteristic may both provide improved bonding between the first material and the third material as well as providing a clear delineation between the first material and the third material.

Embodiments in accordance with the present disclosure provide for a system and method to create a patient-specific replacement body part which may then be displayed in manners that allow for easy disassembly and reassembly. Imaging scans may be acquired by modalities such as magnetic resonance imaging (MRI) or computed tomography (CT) and stored as image data in standard DICOM format in order to promote data portability and interoperability. The data may be further processed to a format compatible with a fabrication apparatus such as a 3-D printer or other automated maker in order to directly create medical models or medical replacement parts from the imaging scans. The fabrication apparatus may include a capability of applying multiple materials to a model of the patient-specific body part.

One general aspect includes a method for creation of a patient-specific body part model, the method including: receiving into a server an image of a patient's internal body part, said server including a processor, a memory, and instructions executable on command, said server capable of logical communication via a communications network; based on the image of the internal body part, generating a digital model of the internal body part; transmitting the digital model via the communications network to a fabrication apparatus in logical connection with the communications network; generating a patient-specific design based upon the digital model of the internal body part and data values of medical imaging studies of the patient; and based on the patient-specific design, fabricating a physical patient-specific body part model including a first material and a second material, the first material including a first material characteristic and the second material including a second material characteristic; and forming an intervening layer including the second material surrounding at least a portion of the first material.

Implementations may include one or more of the following features. The method where the first material includes a Stratasys Vero family material and the second material includes a mixture of Stratasys Agilus family material and Stratasys Vero family material. The method where the third material is another intervening layer including a mixture of Stratasys Agilus family material and Vero family material, where the third layer includes a higher concentration of Agilus family material than the second layer. The method where the first material includes Stratasys Veroclear and the second material includes a mixture of Stratasys Veroclear and Verocolor. The method where the third material includes a magnetic material. The method where the third material includes a metallic stud or pin. The method where the first material includes one or more of a Stratasys Vero family material or a Stratasys Agilus family material and the second layer is a deposit of the first material, where a monomer form of the first material is adhered to the third material before it is affixed to the patient-specific body part. The method where the first material includes titanium and the second third material includes a medication-infiltrated bioresorbable substrate.

One general aspect includes a model of a patient-specific body part including: a first layer of the patient-specific body part including a first material, where the first layer has been fabricated according to a first digital model, where the first digital model has been created with an algorithm performing a calculation utilizing at least a first input of a medical imaging database including data values of medical imaging studies of a patient, where the calculation is performed upon a server having a processor and a memory, the memory storing instructions to be executed by the processor, the server coupled to a user terminal and to a communication network; a second layer of the patient-specific body part including a second material, where the second layer coats at least a first portion of the first layer of the patient-specific body part; a third layer of the patient-specific body part including a third material, where the third layer is affixed to the second layer; and where the second layer forms an intervening layer of the model.

Implementations may include one or more of the following features. The model where the third material includes a magnetic material. The model where the third material includes a metallic stud or pin. The model where the first material includes one or more of a Stratasys Vero family material or a Stratasys Agilus family material and the second layer is a deposit of the first material, where a monomer form of the first material is adhered to the third material before it is affixed to the patient-specific body part. The model where the first material includes titanium and the second third material includes a medication-infiltrated bioresorbable substrate.

One general aspect includes an apparatus for fabricating a model of a patient-specific body part model for a natural body part including: a server having a processor and a memory, where the memory stores instructions to be executed by the processor, and where the server is coupled to a user terminal and to a communications network, where the server stores at least a portion of a medical imaging database including data values of medical imaging studies of the patient; a fabrication apparatus coupled to the server through the communication network, the fabrication apparatus capable of adding multiple materials to a work piece; and a preform of a first material with a surface upon which an intervening layer and a third material is printed.

Implementations may include one or more of the following features. The method where the first material includes titanium and the second third material includes a medication-infiltrated bioresorbable substrate.

In the following sections, detailed descriptions of examples and methods of the disclosure will be given. The description of both preferred and alternative examples though through are exemplary only, and it is understood that to those skilled in the art that variations, modifications, and alterations may be apparent. It is therefore to be understood that the examples do not limit the broadness of the aspects of the underlying disclosure as defined by the claims.

### BRIEF DESCRIPTION OF THE DRAWINGS

The accompanying drawings, that are incorporated in and constitute a part of this specification, illustrate several embodiments of the disclosure and, together with the description, serve to explain the principles of the disclosure:

FIG. 1 illustrates a system to create a patient-specific medical model, in accordance with an embodiment of the present invention;

FIG. 2 illustrates a method to create a patient-specific medical model, in accordance with an embodiment of the present invention;

FIG. 3 illustrates a system to design a patient-specific medical model, in accordance with an embodiment of the present invention;

FIG. 4 illustrates a method to design a patient-specific medical model, in accordance with an embodiment of the present invention; and

FIG. 5 illustrates a method to create a patient-specific medical model, in accordance with an embodiment of the present invention.

FIGS. 6A-6C illustrate different views of intervening layers incorporated into a medical model in accordance with an embodiment of the present invention.

FIG. 7A-7B illustrate different views of a sectioning of a medical model using intervening layers in accordance with an embodiment of the present invention.

FIG. 8 illustrates an exemplary method for creating patient-specific body parts in accordance with the present disclosure.

### DETAILED DESCRIPTION

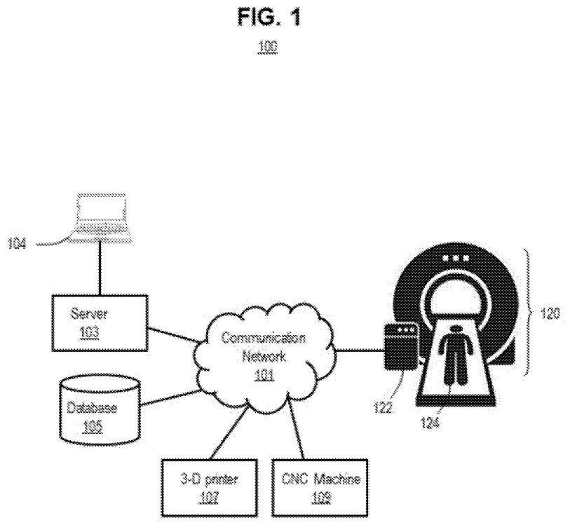

FIG. 1 illustrates a system 100 to design a patient-specific medical model. System 100 includes an MRI machine 120, illustrated as accommodating a patient 124 laying on a gurney and about to undergo MRI examination. MRI machine 120 may include a local control console 122, by which the local operation of MRI machine 120 may be controlled. For example, at local control console 122, an MRI operator (not shown) may control local operation such as movement of the patient gurney to move patient 124 into and out of MRI machine 120, starting and stopping other functions or operating modes of MRI machine 120, etc. An MRI operator may be a human technician or an automaton. Although FIG. 1 illustrates MRI machine 120 as the modality, other modalities such as a CT machine are contemplated as being compatible with system 100.

MRI machine 120 communicatively interfaces with a communication network 101. Communication network 101 may include, e.g., at least a portion of an Ethernet network, a wireless network, or a cellular network, the design of each of which is well known to persons of skill in the art of data networking. The MRI machine 120 and other elements of system 100 may interface with communication network 101 may be wired (e.g., CAT-6 cable) or wireless (e.g., Wi-Fi in accordance with standards such as IEEE 802.11). Communication network 101 may represent various configurations such as more than one connected networks (e.g., multiple peer local area networks (LANs)), or networks of differing hierarchies (e.g., one or more LANs coupled to a wide area network (WAN)).

A server 103 may be coupled to communication network 101. Server 103 may be used by a system operator at terminal 104 to control operation of system 100. Terminal 104 may serve as an interface with server 103 and may be coupled directly to server 103 as illustrated, or terminal 104 may be coupled through communication network 101 to server 103. Controlling the operation of system 100 may include, e.g., remote configuration of MRI machine 120 specific to the requirements for patient 124 (e.g., configuring the number of scans, field strength, duration of each scan, portion of the body to scan, scan plane, distance between adjacent scans, time between successive scans, data formatting requirements, etc.), and being able to study measurements made by MRI machine 120 and/or computations based upon the measurements. For example, an application program hosted on server 103 may render a motion picture to illustrate time-based or position-based changes in successive scans.

System 100 further may include a database 105, in which MRI scans may be stored for analysis. The data may be stored in a DICOM compatible format. In some embodiments, personally identifiable information (e.g., patient name) may be deleted or not saved with scan data. In such embodiments, an identification number (e.g., patient ID number) may be saved with scan data to associate scan data with the correct patient. Database 105 may represent a single database, or a distributed database such as a drive array, cloud storage, or blockchain.

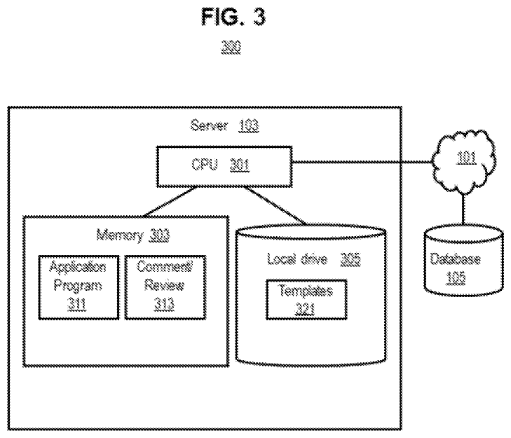

System 100 further may include fabrication apparatus or subsystems to create physical models based upon scan data. For example, FIG. 1 illustrates a 3-D printer 107 and computer numeric control (CNC) machine 109 coupled to communication network 101. Server 103 may include an application program that converts scan data, saved in DICOM format, into a data format and/or file format compatible with a selected fabrication apparatus, such as a standard triangle language (STL) format for 3-D printer 107. Such an application program is described in greater detail with respect to FIG. 3. The fabrication apparatus may create a 3-D model at full size (i.e., life size), or at a selectable percentage of full size for examination purposes (e.g., 50% for a large body part, or 200% for a small body part). A 3-D model to be used as a replacement body part may be ordinarily produced at 100% of full size, though in some embodiments it may be more practical to choose a different size multiplier. Other types of fabrication apparatus also are within the scope of FIG. 1.

As an example of a use-case of system 100, suppose a detailed patient-specific medical model of the liver is needed. For example, the patient may be under consideration for liver surgery, such as to remove cancerous tissue, to remove damaged portions (e.g., damaged by cirrhosis), to repair damage such as a torn liver, to perform a liver transplant (as either a donor or recipient), and so forth. In particular, a multi-layered model of the liver may be needed, e.g., a model of the patient-specific liver with blood vessels, a model of the patient-specific liver with nerves, and so forth. Not all such models may require all blood vessels, nerves, etc. that are present in the actual liver. These separate models may be helpful for different types of surgery, or for different phases of a lengthy surgery. The patient-specific model may be augmented with features to improve the usability or the stability of the model, such as by inclusion of physical support features. Physical support features may be rendered or produced in a way to include a feature not seen naturally. For example, in some embodiments, support features may be rendered in a physical cross-sectional shape of polygons (such as squares, hexagons, octagons, etc.) to differentiate the physical support features from natural features of the patient-specific medical model.

In some embodiments, physical support features may include features that are usable to couple together different portions of the model. For example, physical support features may include a detachable attachment feature, such as a hook-and-loop fastener (e.g., Velcro®). The feature may be produced directly by a fabrication apparatus (e.g., 3-D printer 107 prints the hook-and-loop fastener), or the fabrication apparatus may leave a surface or the like where a hook-and-loop fastener produced separately may be applied manually.

In some embodiments, a patient-specific medical model may be made from a highly flexible material, e.g., a material whose flexibility mimics the flexibility of (or conversely, the stiffness of) the actual body part that the patient-specific medical model is intended to represent. For example, a patient-specific medical model of blood vessels (either arteries or veins) or the lymph system may be fabricated in a double-wall or double-layer process. A first wall or layer (generically, “first layer”) may be fabricated in the shape of the vessel from a relatively stiffer material. A second wall or layer (generically, “second layer”) then may be fabricated adjacent to the first layer (usually, but not necessarily, exterior to the first layer), and being fabricated in a way that is conformal to the first layer. A material used for the second layer may be relatively more flexible than the first layer. After the second layer has been formed, the first layer optionally may be removed (such as by dissolving by a solvent such as acetone that does not significantly affect the second layer), thereby leaving the second layer as a highly flexible patient-specific medical model of the blood vessels.

In some embodiments, a patient-specific medical model made by a fabrication apparatus may have colors selected to provide emphasis to certain features. For example, a 3-D model of a patient-specific model of a heart may be captured at a predetermined point in the heartbeat cycle, and may have clogged portions of various veins and arteries prominently colored if the purpose of the heart model is to plan an open-heart surgery, during which the clogged portions will be treated.

In other embodiments, a specific synthetic body part may be printed from a patient-specific medical model in order to use the synthetic body part as a replacement for a natural body part, such as one that is diseased, damaged, congenitally missing or disfigured, has to be replaced with a larger version as a juvenile patient grows, etc. A synthetic body part may be designed using a material that at least mimics observable or experiential characteristics of the natural body part and may surpass the natural body part if appropriate. For example, synthetic body parts to replace cartilage-based body parts such as ears or the nose may be mimic the flexibility of the natural body parts. On the other hand, synthetic body parts to replace orthopedic body parts such as elbows, kneecaps and spine vertebrae may have a Young's modulus or other indicator of strength, hardness, or other desirable characteristic suitable to function as a replacement body part. Synthetic body parts to replace a joint (e.g., knee or hip) may be made from materials having increased performance, such as having reduced friction during movement or increased tensile strength, as compared to the natural body part.

In some embodiments, imaging scans used to produce a patient-specific medical model may have been gathered with some disparateness in technical aspects of the imaging scans, e.g., gathered or saved with different data types, formats, intake protocols, varying levels of data validation and of anonymization of protected health information (PHI), and so forth. PHI is known as sensitive health information (e.g., a diagnosis of a psychiatric condition) that is personally identifiable with a specific person, and anonymization of PHI is known as the obscuring of data such that a risk of re-identifying the patient from the protected health information has been reduced below a configurable threshold. Disparities in the technical aspects may have arisen if, e.g., different MRI machines 120 were used (e.g., first at a trauma hospital and later at a reconstruction hospital), or if scans were taken in separate sessions in MRI machine 120, etc. Data validation scripts executing on server 103 may be used to convert the disparate scan data into a common (i.e., shared) format so that the scan data is similar enough to be able to be shared and combined into an improved overall patient-specific medical model.

FIG. 3 illustrates portions 300 of system 100 at a lower level of abstraction. In some embodiments, memory 303 of server 103 includes an application program 311 in the form of executable instructions that, when executed by central processing unit (CPU) 301, may perform a conversion of data gathered from MRI machine 120 into models and instructions that can be used by a fabrication apparatus (e.g., 3-D printer 107 and/or computer numeric control (CNC) machine 109, or the like) in order to create a patient-specific medical model. In particular, application program 311 may be used to design a patient-specific customized therapeutic part that can later be fabricated on a fabrication apparatus as a three-dimensional (3D) physical model of patient-specific anatomy, for use as a customized therapeutic part, to provide therapy, or for other medical treatment. A customized therapeutic part also may include portions that are assembled from separate pieces that are not necessarily created on the fabrication apparatus, e.g., pieces that may provide an adhesive, a weave, or a physical trait or material composition that may be difficult to duplicate with fabrication apparatus of the present art, or pieces that may be widely and inexpensively available from generic stock materials (e.g., wire, thread, filament, etc.). A choice of creating a portion of a customized therapeutic part by use of a fabrication apparatus or by assembly from separate pieces may be pre-configured or be controlled by a system operator. Assembly itself may be performed by a skilled human technician or automaton.

Server 103 further may include a local drive 305, which in turn may store templates 321. Templates 321 may be used as a library of standard models for generic body parts, i.e., body parts that are not patient-specific and not necessarily derived solely from data obtained by MRI machine 120 or about patient 124. Standard models of generic body parts may be used when a patient-specific model is not needed for all body parts in a model. For example, if a model is needed for repairing knee ligaments, a standard model may be used to represent a knee cap. In some embodiments, a library of standard models for generic body parts may be created by the system 300 by creating an “average” body part, aggregated from one or more scans of that body part across various patients (and in some embodiments, those scans have all PHI redacted). For example, if a hospital uses the present invention to create 100 patient-specific knee caps, template 321 may comprise a generic knee cap created from the aggregate of one or more of the 100 patient-specific knee caps. By way of non-limiting example, the aggregation may proceed according to size, topography, or relative location of various key parts of the body part.

In some embodiments, portions of system 300 may represent elements accessible over a network 101 via remote desktop protocol. In some embodiments, portions of system 300 may represent an elastic cloud-based computing environment, such as VMware®, Amazon Web Services®, Microsoft Azure®, and so forth.

Memory 303 of server 103 further may include an application program 313 in a form such as executable instructions that, when executed by central processing unit (CPU) 301, may allow for the review, comment and editing of a model for a patient-specific customized therapeutic part, prior to its fabrication by a fabrication apparatus.

Embodiments in accordance with the present disclosure allow for review and practice of a medical procedure (e.g., a surgery) by a doctor, prior to performing the medical procedure on a live patient. Having a patient-specific customized therapeutic part also may facilitate additional rounds or levels of medical review before a medical procedure is performed on a patient. For example, the patient-specific customized therapeutic part may be used to consult with a more knowledgeable or experienced specialist, or to use for a peer review of a proposed surgery, and so forth. The consultation or review may be performed locally or remotely. The patient-specific customized therapeutic part also may be used to explain a proposed medical procedure to a patient, in order to better explain the procedure to the patient and in order to obtain a more informed patient consent.

The medical procedure also may involve replacing a body part with the patient-specific customized therapeutic part. In some circumstances, when a patient-specific customized therapeutic part is intended to be used for multiple purposes (e.g., to explain a procedure, and to use as a replacement for the body part), separate models may be fabricated. For example, for the purpose of explaining a procedure, a patient-specific customized therapeutic part may emphasize or make more prominent (e.g., by use of a bold color) any unusual anatomy or anticipated problems. On the other hand, a patient-specific customized therapeutic part intended for inclusion in a patient may be fabricated from more inert materials, or from a material that is relatively more biologically compatible with human or animal tissue characteristics.

In some embodiments, a patient-specific customized therapeutic part may include support features to support other portions of the part. Such support features may be helpful to increase a robustness of otherwise delicate or fragile features. In some embodiments, a support feature may be constructed with at least a portion being external to a second portion of the patient-specific customized therapeutic part, e.g., provided as wires, threads, filaments, adhesive surfaces, and so forth. In other embodiments, support features may be constructed with at least a portion being internal to the patient-specific customized therapeutic part, e.g., by use of a relatively stiffer material than the natural counterpart. A stiffer material may be used in concealed locations, e.g., on at least a portion of a concealed surface such as an interior of a vessel, bronchial tube, or other tube-like structures. A concealed surface also may be provided as an interior layer, similar to a fascia, or an outer surface similar to a shell. Such support features may be more useful for patient-specific customized therapeutic parts intended to be used for demonstrative purposes rather than to be used in vivo in the patient.

In some embodiments, the material used for the support surface may be selected to provide a selectable amount of support. For example, the support surface may have varying degrees of rigidity, pliability, softness, flexibility, and so forth. The support surface also may derive at least a portion of its support characteristics from being formed in a rigid lattice-like shape, such as a triangular or hexagonal tessellation.

In some embodiments, the support features may be visibly coded (e.g., by use of a non-natural color) in order to distinguish the support features from the natural portions of the patient-specific customized therapeutic part.

Embodiments in accordance with the present disclosure may include a continuation of anatomical features. For example, a continuation of an anatomical feature may be user-designated, such as made electronically on an editable MRI scan or on a computer model generated based upon normalized data from multiple MRI scans. Examples of such continuations may include blood vessels, nerves or other anatomical features, or proposed artificial items (e.g., stent, pacemaker, cochlear implant, brain stimulus probe, etc.) as they would be included in and around anatomical features. Such features may be AI designated and color coded to distinguish the feature from the rest of the patient-specific customized therapeutic part. Such features also may have a contiguous material and/or surface quality. As with support features described above, the continuation may be fabricated with a selectable amount of tactile characteristics such as varying degrees of rigidity, pliability, softness, flexibility, and so forth. The continuation also may derive at least a portion of its tactile characteristics from being formed in a rigid lattice-like shape, such as a triangular or hexagonal tessellation.

Embodiments in accordance with the present disclosure also may facilitate user control of a three-dimensional model used by the fabrication apparatus. For example, embodiments may allow a user to select a portion of a scan or a model, for generation as a physical representation of the patient-specific customized therapeutic part, based upon normalized data from multiple MRI scans.

Embodiments may allow a user to print selected portions of a patient-specific customized therapeutic part. For example, a user may be allowed to pan, rotate, zoom, etc. with respect to selected portions of the patient-specific customized therapeutic part in order to better illustrate a portion of greater interest. Some embodiments may fabricate outer layers of the patient-specific customized therapeutic part in a transparent or translucent material, in order to make at least partially visible an interior structure of interest. In some embodiments, an outer layer may comprise a material easily dissolvable in a solvent like acetone or water. Some embodiments may fabricate portions of the patient-specific customized therapeutic part in special colors. For example, a cancerous organ may be rendered with the cancerous portion of the organ rendered in a different color to visually highlight it. In some embodiments, the patient-specific customized therapeutic part may omit one or more sections, portions, features or the like in order to better illustrate features of interest. For example, a patient-specific customized therapeutic part fabricated as a bone in order to illustrate a bone marrow transplant procedure may be fabricated in cutaway form, such that the marrow interior of the bone is visible.

Embodiments may design a patient-specific portion of the body, not limited to a single organ. For example, embodiments may include one or more features in a given area of the body, multiple specific organs (e.g., related organs like the liver, gall bladder, and pancreas), bones with attached ligaments, etc. In some embodiments, features of less interest may be omitted in order to concentrate attention on the features of greater interest.

In some embodiments, aggregated scan and build data may be analyzed to produce an improved patient-specific medical model. For example, artificial intelligence (“AI”) analysis techniques may be used to analyze the injury or disease state of a body part to be modeled or to be replaced, using an AI knowledgebase stored in database 105. The AI knowledgebase may include, e.g., average or typical scans or the same, similar, or related body parts from other patients, to be used for comparison sake.

This may be useful to produce an AI-guided model of therapeutic treatments, e.g., for a specific patient and based upon the patient's specific medical model, produce a prediction of success rate and possible complications from various modeled therapeutic treatments. For example, the AI-guided model may be able to model one or more predicted outcomes of surgery or no surgery (e.g., for orthopedic injuries), predicted treatment results over time (e.g., how soon after surgery a benefit will be observed, and how long that benefit will last after the surgery), and how the state of a disease or injury will advance over time (e.g., arthritis). These results from the AI-guided model may be compared to equivalent predictions based on professional judgment from a healthcare professional (e.g., a physician rendering an opinion within the area of their expertise) in order to improve over time the quality of the AI-guided model and/or the professional judgment.

Embodiments in accordance with the present disclosure facilitate the selection of materials used in 3D models. For example, material characteristics or material features of the materials may be matched to corresponding characteristics or features of the replacement body part. Material characteristics and material features may include hardness, softness, firmness, flexibility, tackiness, slipperiness, smoothness, roughness, friction or resistance to wear (especially for joints), ductility, brittleness, density, color, porosity, tempered, annealed, tensile strength, molecular or microstructure characteristics (e.g., grain, fibers, nanolattice), thermal resistance, hydrophobic or hydrophilic, radiopacity, and so forth. Certain material characteristics may be avoided when possible. For example, usage of a material attracted to a magnet (e.g., a steel rod) may be avoided. Such materials may be problematic in the presence of magnetic fields (e.g., at a metal detector, in an MRI machine, etc.).

Specific anatomical parts, organs, features, etc., may be characterized in terms of body part characteristics, and the body part characteristics may be associated with the closest available material characteristics or material features. Alternatively, a body part characteristic may be associated with at least a threshold level of a corresponding material characteristic or material feature. Alternatively, a body part characteristic further may be associated with a material having preferably a greater amount of the material characteristic or material feature. For example, a knee cap may be characterized by a certain Young's modulus. It may be desirable to associate a material with its Young's modulus. In some embodiments, it may be desirable to choose a material with a Young's modulus approximately commensurate with that of a knee cap; in other embodiments, it may be desirable to choose a material with a greater or lesser Young's modulus than that of a knee cap, based upon a performance characteristic chosen by one or both of an administering healthcare provider and a patient involved.

By way of further example, bone may be characterized by an amount of stiffness, or an amount of stiffness per unit cross-sectional area. A major weight-bearing bone may such as a tibia, a femur, or a vertebra in the lower back may have an amount of stiffness that is greater than the stiffness of a non-weight-bearing bone such as a collarbone, a rib, a metacarpal or phlanges bone, etc. A replacement bone part may be made from a material having at least as much stiffness as the corresponding natural bone, and preferably a greater amount of stiffness.

In another example, a replacement joint may have an amount of friction that is less than the friction of a corresponding natural joint, and/or a resistance to wear that is greater than the resistance the wear of the corresponding natural joint. For such a body part, a greater amount of a desirable characteristic (e.g., resistance to wear) is better, and a lesser amount of an undesirable characteristic (e.g., friction) is better.

For some body parts (e.g., an external body part having at least a portion as an external skin surface), it may be preferable to mimic a characteristic of a natural body part as closely as possible by appropriate selection of material rather than to exceed the characteristic of the natural body part. For example, a prosthetic hand (or ear) preferably should have a texture, softness, firmness, flexibility, tackiness, etc., that matches a natural hand (or ear). Consequently, a silicone or similar material may be useful for an external body part. As a counterexample, a prosthetic hand that is too stiff may be perceived as being claw-like.

For some body parts, it may be preferable to select a material color that matches the color of the original body part. For example, it is more important to match a color of a visible body part such as an ear or prosthetic hand, compared to the color for an internal body part such as a bone, an organ, a stent, etc. In some embodiments, a non-natural color may be used for an internal body part in order to easily identify it as an artificial part during any future surgeries. In some embodiments, several different colors may be used for external body parts because skin tone is not one consistent color but a mixture of textures and colors. Skin around the knuckles, for example, usually has more red pigment, as do palms. The palm of the hand is also commonly lighter in color than the back of the hand and the arm.

In some embodiments, a body part model to be used for illustration or teaching purposes, but not for usage as an operational body part, may be fabricated from a material having a different material characteristic or material feature than the corresponding natural body part. For example, a model of an internal organ (e.g., liver, kidney, etc.) may be fabricated with a non-natural color in order to highlight its position within the torso.

In some embodiments, an application program such as application program 311 may be used to help create the patient-specific body part. For example, application program 311 may have access to one or more of a library of material characteristics for various fabrication materials, and a library of normal characteristics for various natural body parts. The one or more libraries may be stored in a persistent memory such as database 105 or local drive 305. Application program 311 may offer to a user a selection of materials to use when fabricating the patient-specific body part. A best match or suggested match may be offered, based upon a comparison of at least one material characteristic with normal or typical characteristics for the body part. In some embodiments, the selection may be modified by specific abnormal characteristics of a patient-specific body part. For example, if a patient-specific model of a liver scarred with cirrhosis or a skin surface scarred with psoriasis is desired, the material selection for fabrication may be modified based upon the condition the model should include. A selection of fabrication materials may be made by a user using familiar user interface GUI controls such as a drop-down selection list. The selection of fabrication materials also may be influenced by the physical size of the model being produced. For example, if a large portion of the body is being produced, or a relatively smaller portion (e.g., an organ) is being produced at a larger-than-life size, then additional physical support features may be needed. Preferably, any additional physical support features will be provided in a way that is not readily visible to a casual observer.

In some embodiments, fabrication of a model may depend upon a user-controlled quality setting. For example, a high-quality setting may produce a model having superior realism for as many body part characteristics as possible. Such a model may be slower to produce or may use more expensive materials. In other circumstances, when a faster or less expensive model is sufficient, a model may be produced with a lower quality setting.

In some embodiments, suitable materials for a replacement body part and/or a body part model may include one or more of silicone, plastic, metal, ceramic, and so forth. All materials should include biocompatibility with human tissue. In some embodiments, a replacement body part such as artificial skin may be fabricated at least in part with a culture of human cells or compatible animal cells (e.g., pig cells) grown on a substrate mesh or membrane.

In some embodiments, a body part model may be fabricated from a luminescent material in order to facilitate illuminating at least a portion of the body part model. In some embodiments, luminescent portions of a body part model may be designed in coordination with light emitting diodes (LEDs) or other lighting sources. For example, LEDs may be either embedded within the body part model as internal illumination, or as external illumination of the body part model, or a combination of both lighting locations. In some embodiments, the body part model may be fabricated to be compatible with a handheld light source, e.g., by having sufficient clearance for the movement of a handheld light source.

In some embodiments, when a replacement body part is being fabricated for use by a patient, a choice and selection of materials may depend upon whether the replacement body part is intended for internal usage (e.g., as orthopedic repair) or for external usage (e.g., as orthopedic support). In some embodiments, a replacement body part and a body part model may be fabricated either sequentially or simultaneously (subject to fabricator apparatus availability), from the same patient-specific medical model.

In some embodiments, when a body part model or a replacement body part requires a fastener (such as a screw for, e.g., a dental implant) to secure together pieces or to secure the part to a bone or similar rigid part, application program 311 may design the placement and size of the fastener, and/or a place for the fastener to attach (such as a threaded screw hole). The fasteners themselves may be conventionally-supplied medical grade fasteners or may be printed concurrently or in parallel with the body part. Fasteners may be generated specific to a particular procedure. For example, a fastener may have a length, width, and/or thread pattern specific to a size and shape of a 3D-manufactured body part.

In some embodiments, application program 311 may facilitate a selection of FDA-approved parts based upon the patient-specific medical model, provide a list of any additional required parts in order to verify that the necessary parts and components are on hand and available before a surgery. Doing so helps allow for peer review prior to surgery, helps obtain more informed patient consent prior to surgery, and may be able to save time during the surgery itself.

Embodiments in accordance with the present disclosure are not limited to the creation of a patient-specific medical model only for use as a replacement body part or for illustration during training or instructions. Embodiments are also useful to create a patient-specific medical model for use as, e.g., prototypes, final use parts, and emergency usage parts (possibly on a temporary basis) in trauma or triage situations. Fabrication apparatus and its usage will comply with applicable law and regulation, and also preferably adhere to best practices, with respect to: equipment operator identification, qualifications, and training; qualifications and certifications such as ISO 9000; maintenance requirements, logs, and schedules; and material chain of custody such as material sourcing, material quality assurance (QA), and material storage.

Manufacturing QA, including monitoring and observation, may be performed with technological apparatus where feasible, such as video monitoring and recording or other sensors to detect flaws or assure compliance of individual parts with a design, and provide real-time observation capability to a system operator. The fabrication apparatus may undergo routine maintenance such as scheduled maintenance, cleanings, minor repairs as needed, disposal of replaced parts. The fabrication apparatus may maintain records thereof including at least some of: operator or technician identification, title, dates, etc. Maintenance tools, equipment, and methods may include: usage of clay tools and brushes; pressure washing; usage of pumps, tanks or buckets; ultrasonic cleaning; sterilization (e.g., by heat, or chemicals such as alcohol or bleach); and so forth. Cleaning and maintenance procedures may maintain sterility, adhere to biohazard standards when and where applicable, and dispose of waste in a manner commensurate with its risk. If necessary, cleaning and maintenance procedures may be performed by guided or imaging tools, such as a remote-controlled cleaning robot. Where feasible and without creating a biohazard, waste materials may be recycled in accordance with prevailing technology, standards, and initiatives.

In some embodiments, a body part model may be fabricated with an aesthetically pleasing appearance (e.g., a glossy finish) or physical texture (e.g., smooth). In some embodiments, it is preferable that the aesthetic artifacts (e.g., surface reflectivity or surface micro-texture) are not important to the demonstrative purpose of the body part model. For example, if a body part model of a liver is used only to illustrate the location of the liver in the abdominal cavity and its placement with respect to other internal organs, then the surface reflectivity and surface micro-texture of the liver model is not important. On the other hand, if the body part model of a liver is intended to function as a replacement liver within the body of a patient, then surface micro-texture of the liver model may be critical. Standard model-making tools and methods may be used to provide an aesthetically pleasing appearance, such as sanding (e.g., grits of 100-5000), polishing and polish compounds, polishing cloths and sponges, etc. Power tools (e.g., oscillatory or rotary saws, sanders, etc.) may be used. Priming (e.g., heat or chemical) and painting (e.g., spray, brush, roller, airbrush, etc.) may be used.

The fabrication apparatus, its usage, its interface to other portions of system 100, and its scheduling may be automated to the extent feasible (e.g., if justified from a cost/benefit analysis) compared to alternative methods such as a manual method.

In some embodiments, post-process validation may include a manual measurement to verify that parts are being fabricated according to design. For example, validation may include parts scanning (e.g., a laser-based 3D measurement), and a computational comparison of the measurement to the design. Machine learning techniques may be used, e.g., if a systematic bias is found in the 3D measurements. Token verification may also be utilized.

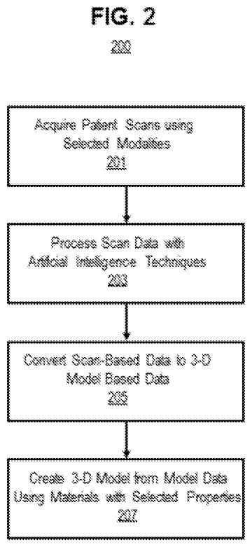

FIG. 2 illustrates a process 200 to design a patient-specific medical model in accordance with an embodiment of the present invention. Process 200 begins at step 201, at which patient-specific scan data is acquired using a selected modality, e.g., by CT scan or by MRI scan. Process 200 continues to step 203, at which artificial intelligence techniques optionally may be applied to the scan data in order to improve the data quality, and to analyze the injury or disease state of body part to be modeled or to be replaced.

Next, process 200 may transition to step 205, at which the scan-based data of previous steps may be converted into a data format usable by a fabrication apparatus or subsystem, such as 3-D printer 107 and computer numeric control (CNC) machine 109, to design physical models based upon scan data. For example, languages, models and data formats used by a fabrication apparatus may include one or more of G-code, STereoLithography (STL), AMF, ReplicatorG, Skeinforge, and Cura.

Next, process 200 may transition to step 207, at which a 3-D model may be designed from the physical model data of step 205, using materials with selected properties.

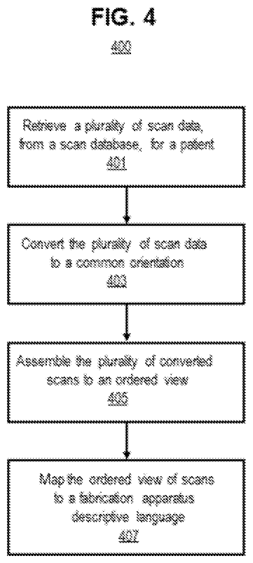

FIG. 4 illustrates a process 400 that may be used to design a patient-specific replacement body part. Process 400 may begin at step 401, at which a plurality of scan data, from scan database 105, may be retrieved for a patient. Next, process 400 proceeds to step 403 at which server 103 may convert the plurality of scan data to a common orientation. For example, the scan data may be converted to a predetermined angular view, resolution, size, and so forth. Next, process 400 proceeds to step 405 at which server 103 may assemble the plurality of converted scans to an ordered view. For example, the converted scans may be reordered to a sequential set of slices from top to bottom of a knee joint. Next, process 400 proceeds to step 407 at which server 103 may map the ordered view of scans to a fabrication apparatus descriptive language. Next, at step 408, machine control sequences may be generated. Next at step 409 the resulting control sequences may be transmitted to a fabrication machine. Transmission may occur via a communications network, wired connection, or any other suitable data transfer method. Proceeding next to step 410, one or more three-dimensional printing devices may be activated. Next, at step 411, multiple disparate pieces may be produced. In some embodiments, the disparate pieces may be joined together by intervening layers. In some embodiments, one or more of the disparate pieces may comprise a material soluble in a given solvent.

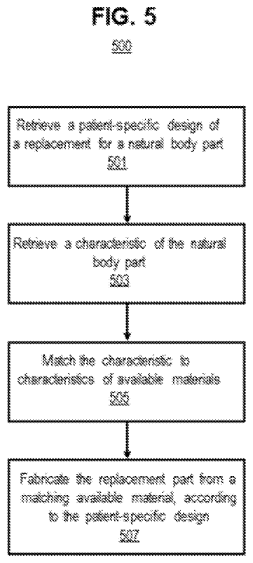

FIG. 5 illustrates a process 500 that may be used to design a patient-specific replacement body part. Process 500 may begin at step 501, at which a patient-specific design of a replacement for a natural body part may be retrieved. The design may be retrieved from a non-volatile memory such as database 105, and the design may be created by an application such as application program 311, using data obtained from system 100.

Next, control of process 500 progresses to step 503, at which a characteristic of the natural body part may be retrieved. For example, a texture of a patient's natural skin may be retrieved. Additional characteristics of the natural body part may be retrieved, such as color or softness. Specific characteristics that are retrieved may be dependent upon, and appropriate to, the natural body part. As a counterexample, hardness would be unlikely to be appropriate for natural skin.

Next, control of process 500 progresses to step 505, at which the retrieved characteristic(s) of the natural body part is compared with, and matched to, a corresponding characteristics of materials that might be available to fabricate a patient-specific replacement body part. The required closeness of a matching material selected from the available materials may be controlled by a designer using the system, e.g., by setting a tolerance level for the maximum deviation between a characteristic of the natural body part and a characteristic of the available material. Moreover, the tolerance level may vary depending upon a characteristic of a patient. For example, the Young's modulus of the patellar tendon in men is typically 99% greater than the Young's modulus of the patellar tendon in boys. See T. D. O'Brien, et al., Mechanical Properties of the Patellar Tendon in Adults and Children, 43 J. BIOMECH. 1190 (2010). Accordingly, men may be assigned a higher tolerance than boys when creating a model of a patellar tendon.

Next, control of process 500 progresses to step 506, at which the patient-specific replacement body part may be fabricated from the selected matching material, according to the patient-specific design. In some examples, an additional purpose may include replacing or augmenting the characteristics of the natural body part.

Intervening Layers

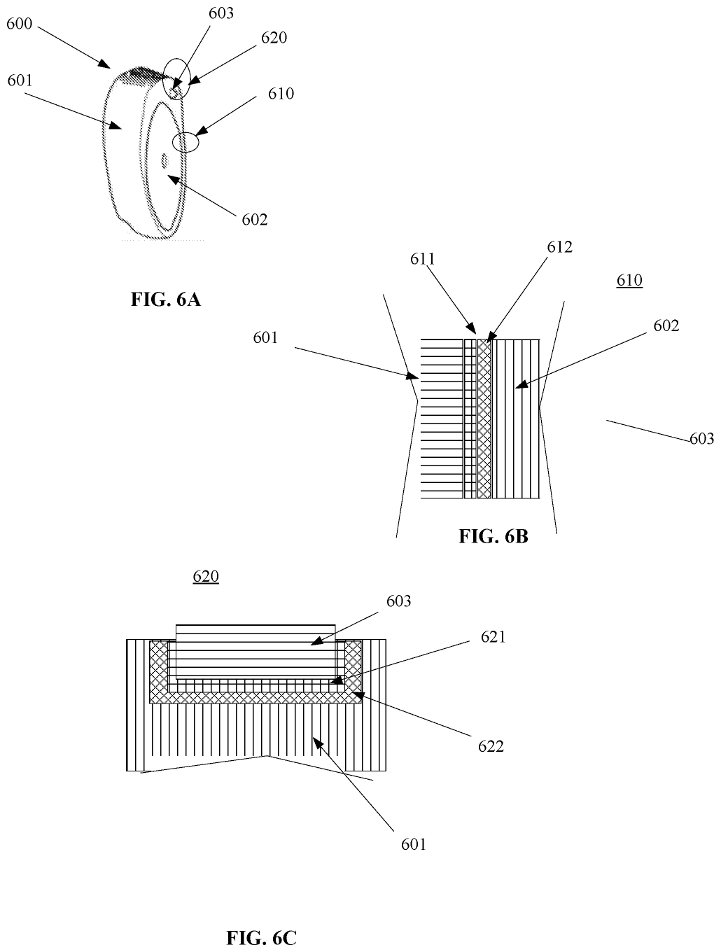

Now that a physical model can be obtained of a patient-specific body part, means of displaying such a three-dimensional object in manners that allow for display of internal structure in cross section are illustrated in reference to FIG. 6A. Modelled structure 600 may represent a portion of a particular organ of a patient that may have been produced from a model generated by a medical imaging study. The model may be produced in multiple sections which cut from an edge to the core of the model. The sections may have multiple regions of different structure. For example, there may be “bone” and “soft-tissue” regions in a section. These different regions may be best depicted with different types of materials used for a three-dimensional structure. In a non-limiting example, soft tissue 601 may be represented by a silicone type material and a harder material such as cartilage or bone 602 may be represented by a hard thermoplastic such as ABS. There may be many different types of materials that have different properties that can convey different aspects of a model. There may also be physical registering devices 603 made of other materials. There may also be sensor and identification devices 605. The combination of different material types in a single model may create functional issues at the interfaces between the materials. For example, adhesion may be difficult, or thermal expansion and contraction may differ and cause stress or the like. In these cases, a superior model may be obtained by printing or otherwise assembling a model with different materials containing, between the major discrete layers, other materials in relatively thin layers that may be considered intervening layers that buffer the changes between the major discrete layers with smaller changes. In the following table examples of major material types with intervening layer examples is depicted.

TABLE 1
EXAMPLES OF MATERIALS OF INTERVENING LAYERS
Generic Resin Deposition Printing Materials

| Trade Name | Common Analog | Notable Variations (Flavors) |
|---|---|---|
| Stratasys Agilus 30 | Rubber-like | Clear, Black, White |
| Stratasys Tango+ | Rubber-like | Clear, Black, Gray |
| Stratasys Vero Family | ABS-like | White, Black, Clear, |
| | | Cyan, Magenta, Yellow, |
| | | Flex, Vivid |
| Stratasys Agilus | Range of Harder | |
| 30 + Vero Mixture | rubbers - Softer | |
| Family | thermoplastics | |
| Stratasys Medical | Biocompatible | MED 610, MED 620, |
| Materials | Plastics and | MED 625FLX |
| | Rubbers | |

| | |
|---|---|
| Base Material (Any may be A or C) | Mixture Description (B) |

Other Generic Printing Materials

| | | |
|---|---|---|
| ABS | Nylon | Material B that consists |
| PLA | Wood | of any Base Material A |
| PVA | Gold | inflitrated with Base |
| Ultem | Copper | Material C |
| Brass | Wax | |
| Silver | Ceramic powder | |
| Glass powder | PET/PETG | |
| PEEK | HIPS | |
| Hemp | Titanium | |
| Stainless Steel | Carbon fiber/ | |
| | micro-carbon | |
| High electrical | | |
| conductivity material, | | |
| i.e.: carbon black, | | |
| carbon nanotubes, | | |
| graphene | | |

Other Generic Interverning Materials

| | | |
|---|---|---|
| Sylgard | Teflon | Flex Seal |
| Silicone | Urethane | Antimicrobials |
| Graphene | Graphene aerogel | Silica aerogel |
| Medication inflitrated | Epoxy | Cyanoacrylate |
| plastics/rubbers/ | | |
| aerogels/polymers/ | | |
| coatings | | |

Referring to FIG. 6B, in some examples, the major layers such as the exemplary soft tissue 601 in Stratasys® Agilus 30 and bone 602 in Stratasys® Vero Family may have a gradated interface with thin layers, which conform to the shape of the interface between the major layers 610. For example, adjacent to the Stratasys® Agilus 30 layer may be a layer comprising a coincidently printed layer of 33% Stratasys® Vero Family material with 67% of Stratasys® Agilus™ 30 layer 612 as a non-limiting example. Accordingly, and also as a non-limiting example, the intervening layer to the Stratasys® Vero Family™ layer may be a layer comprising coincidently printed layer of 66% Stratasys® Vero Family™ material with 67% of Stratasys® Agilus™ 30 layer 611. These layers may form better interfacial layers to each other and to the respective major layers than would occur at an interface between Stratasys® Agilus 30 and Stratasys® Vero Family for example. As shown in Table 1, the spirit of this example has many more examples, where the interface between two different materials may be filled with intervening layers of mixtures of the first material (Material A in the table) and the last material (Material C in the table).

As shown in the bottom of the table, there may be materials that may be used in intervening layers that are not mixtures of the two materials that surround the intervening layers. There may be coatings of these materials applied by printing, spraying, dipping or other means to surround a first model layer with the intervening layer. Furthermore, as in the example with Stratasys® materials, there may be multiple intervening layers chosen from the examples at the bottom of the table which are not mixtures of other materials. Thus, for example, a part printed with FDM ABS material may be dip coated with a layer of Sylgard, the result of which may be a starting substrate for printing to a Stratasys® Vero family material.

Referring to FIG. 6C, in some examples, intervening layers may not only conform to an interface, they may surround a portion of a material. In an example 620, the physical registering device 603 may be coated with a number of intervening layers 621 and 622. These intervening layers may provide for better properties of joining an alignment device to the silicone representation of a soft tissue 601. As mentioned in the previous table, there may be exemplary combinations of intervening layer material choices for various common materials.

Intervening layers may also be used to convey information. Some combinations of basic materials may have appearances or surface properties that are relatively similar to each other. Therefore, a user who is viewing the model may have difficulties perceiving the different layers or their intended different structural representations. Therefore, the presence of an intervening layer may be used to highlight such an interface. For example, a coloration of the intervening layer may convey an interface. In other examples, a surface texture or other property such as tackiness may convey a boundary aspect.

Intervening layers may also include sensors that monitor and transmit information related to the general region of the model that the intervening layer is attached to.

In some examples, one or more intervening layers may surround a feature and may give the surrounded piece a different physical aspect. For example, a hard-slippery material may be coated with an intervening layer which has more tackiness allowing to model sectional pieces to stay better aligned when they are stacked.

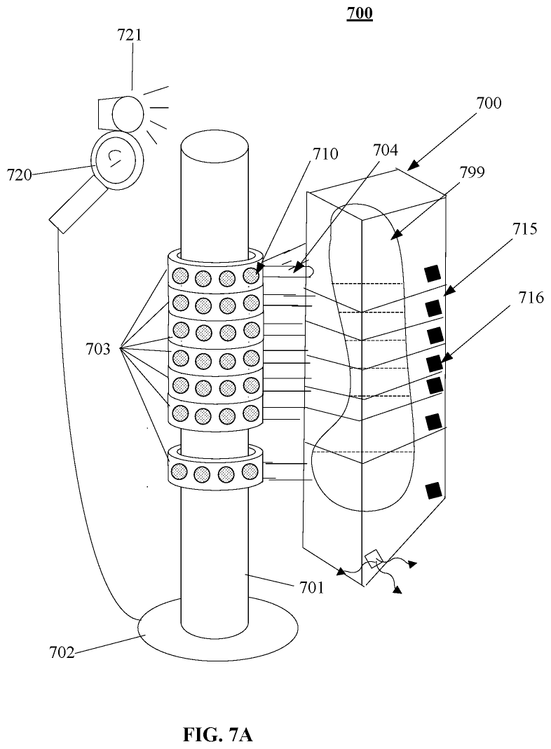

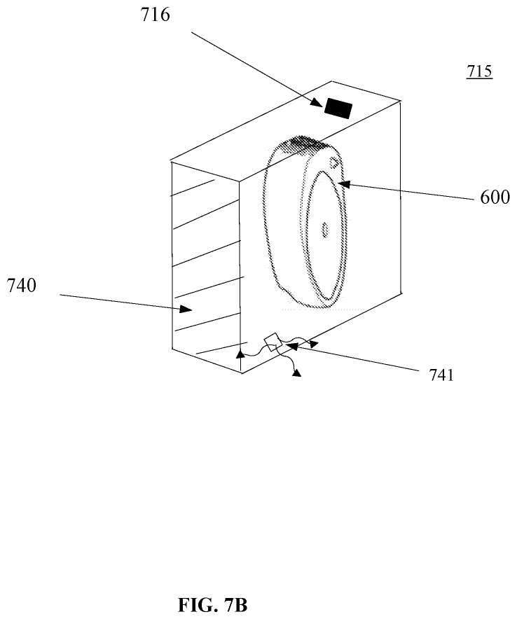

In some examples, an intervening layer or intervening layers may surround model sectional pieces to better protect the fine features of a layer in a more resilient layer. Referring to FIG. 7A, an example of a formed model 799 encapsulated with an exemplary intervening layer of lucite is illustrated as a composite model 700. Composite model 700 may be made of multiple sections 715. The formed model may have a delicate structure that, when encapsulated in lucite, may be readily observed but also supported with an intervening layer of lucite that may then join to external support structures 704. An intervening layer may also include superior physical qualities to augment physical performance.

In the example of FIG. 7A, an axle 701 is supported upon a stand to ultimately support the model. The axle provides vertical location and support for the individual component sections. In an example, fastening pieces 703 may be positioned in the vertical dimension. The individual fastening pieces 703 may have the ability to rotate and may be formed with such features as bearings or other means to allow rotation while holding a vertical dimension fixed.

In some examples, fastening pieces may be covered with a plurality of fastening devices 710 which may be used to hold an individual section piece 715 in place. One or more of the section pieces may have at least one and sometimes two or more fastening arms 704 which can attach to the fastening device and be affixed to an individual sectional piece 715.

In some examples, the models may have a base 702 which is stable and may have articulating abilities. As well the model may have built-in tools such as a magnifying glass 720 and light bulb 721. In some examples, various covers and decorations may be utilized to complete the appearance of the model structure. A section piece 715 may also be removed from the model and inspected as a separated piece. Referring to FIG. 7B, an example of an unfastened piece 740 is illustrated separated from the supporting structure. The various fastening devices may allow for the piece to be easily re-attached and rotated into place within the model body. The fastening devices may include numerous types of devices. An intervening layer, in this example lucite, may surround the internal modelled structure 600.

There may be other features that are imparted or attached to the intervening layer. In an example, visible identification marks 716 may be placed on the intervening layer. These marks may include numbers, characters or other iconic representations that may help a user to understand the alignment of different pieces. In some examples, an identification device may include an RF-ID tag 741 or other device capable of wireless communication between itself and an external transceiver device. Edges of the intervening layer may have coloration 740 imparted to it to help identify the section and its correction orientation in the overall model. There may be numerous other characteristics that are used by the incorporation of an intervening layer or layers to a model such as but not limited to: softness or hardness, tackiness or slipperiness, coloration, opaqueness, transparency and the like. A layer may provide a lubricating surface or alternatively impart a frictional aspect to keep a part from sliding.

As illustrated in FIG. 7A, each of the various components or sections of a model may have intervening layers incorporated upon and/or within their structure. However, in some examples, only a subset of the various components or sections of a model may have such layers incorporated. In some examples, the intervening layers may be added to a model component in sequential discrete processing steps. In other examples, a production process such as a three-dimensional printer may have the capability of adding different layered components to a composite piece during the same processing in a simultaneous or approximately simultaneous manner.

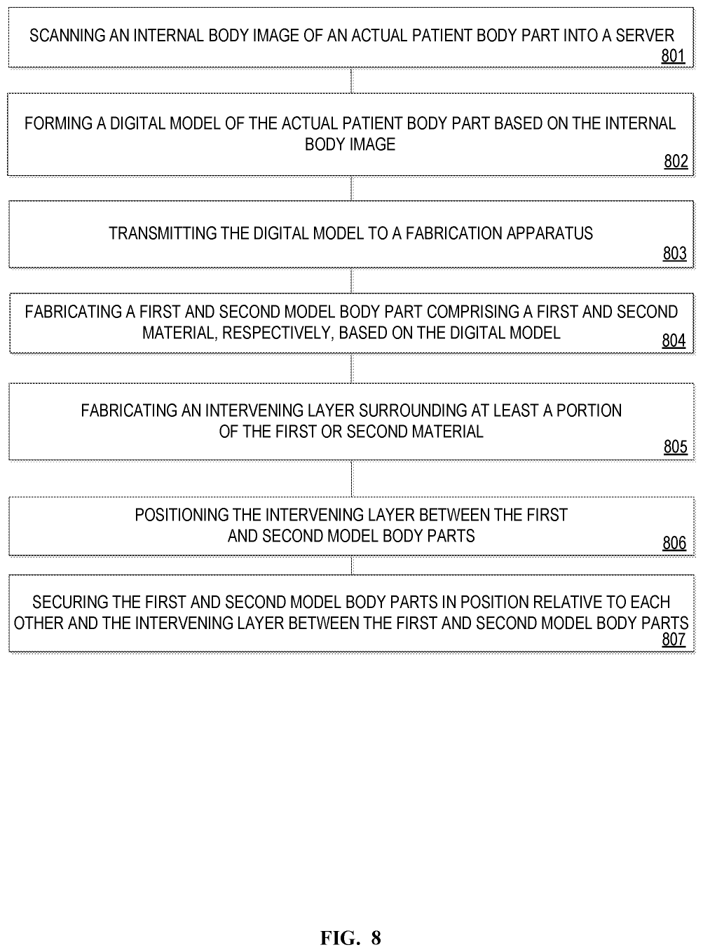

Referring now to FIG. 8, an exemplary method for creating patient-specific body parts is shown, in accordance with the present disclosure. At step 801, an internal body image of an actual patient body part may be scanned into a server. The server may comprise a processor, a memory, and instructions executable on command, and may be logically connected to a communications network. The internal body image may comprise an MRI image, a CT scan, or a CAT scan, as non-limiting examples.

At step 802, a digital model of the actual patient body part is formed based on the internal body image. This formation is done in accordance with the present disclosure. At step 803, the digital model is transmitted via the communications network from the server to a fabrication apparatus, such as a 3D printer. Then, at step 804, the fabrication apparatus fabricates a first and second model body part comprising first and second materials, respectively. The first and second materials may comprise different, identical, or approximately identical substances.

At step 805, an intervening layer surrounding at least a portion of the first or second material is fabricated based on the digital model. This intervening layer is then, at step 806, positioned between the first model body part and the second model body part. Finally, at step 807, the first and second model body parts are secured in position relative to each other and the intervening layer between the first and second model body parts.

Examples of physical models which may be presented in a manner that allows for easy presentation of cross-sectional structure as well as the overall structure examples of medical body parts and other medical related models have been discussed. It may be apparent that the techniques and apparatus as have been described may have general applicability to other types of physical models, particularly those whose internal structure is elucidated by a measurement means which can sense parametric variation across the three-dimensional structure. Accordingly, it will be understood that various modifications may be made without departing from the spirit and scope of the claimed disclosure.

### Glossary

Scan—As used herein, “scan” means a medical imaging study which penetrates the tissue layers of a patient's body to generate a detected signal that reflects characteristics of the patient's body layers with three dimensional reference. Common modalities of the scan may include computed tomography, magnetic resonance imaging, ultrasound, and positron emission tomography as non-limiting examples and the modalities may use contrast agents and dyes to elucidate further structure or three dimensional functional aspects. In some examples, a scan may reflect a three dimensional aspect of the surface layers of the patient's body such as the shape of the skin layers.

Replacement Body Part—as used herein a “replacement body part” refers to a modeled structure created in materials that form a three-dimensional model to demonstrate structural aspects of a patient's organ. In some examples, a structural replacement may be used to demonstrate structure and/or practice surgical procedures. In some examples, the printed body part formed of inorganic material may be used as a replacement piece within a patient. The replacement body part may strengthen a portion of a patient's body, increase some kind of performance aspect of the body part, or it may include sensors to generate data from its location. In some examples, the replacement body part may be located within a patient's body. In other examples, the replacement body part may be located externally to the patient's body.

What is claimed is:

1. A method for creation of a patient-specific body part model, the method comprising:
receiving into a server an image of a patient's internal body part, said server comprising a processor, a memory, and instructions executable on command, said server capable of logical communication via a communications network;
based on the image of the internal body part, generating a digital model of the internal body part;
transmitting the digital model via the communications network to a fabrication apparatus in logical connection with the communications network;
generating a patient-specific design based upon the digital model of the internal body part and data values of medical imaging studies of the patient; and
based on the patient-specific design, fabricating a physical patient-specific body part model comprising a first material and a second material, the first material comprising a first material characteristic and the second material comprising a second material characteristic; and
forming an intervening layer comprising the second material surrounding at least a portion of the first material.

2. The method of claim 1 wherein the second material characteristic improves bonding of a third material in a third layer to the patient-specific body part model.

3. The method of claim 2 wherein the first material comprises a Stratasys® Vero family material and the second material comprises a mixture of Stratasys® Agilus family material and Stratasys® Vero family material.

4. The method of claim 3 wherein the third material is another intervening layer comprising a mixture of Stratasys® Agilus family material and Vero family material, wherein the third layer comprises a higher concentration of Agilus family material than the second layer.

5. The method of claim 2 wherein the first material is titanium and the second material comprises a mixture of titanium and PEEK.

6. The method of claim 1 wherein the second material characteristic provides a visible separation between the first material and a third material.

7. The method of claim 6 wherein the first material comprises Stratasys® VeroClear™ and the second material comprises a mixture of Stratasys® VeroClear™ and Verocolor.

8. The method of claim 6, wherein the second material comprises a mixture of VeroClear and Verowhite and the third material comprises VeroClear™ and fabricating steps comprise stereolithography.

9. The method of claim 1, wherein the internal body image comprises data relating to a magnetic resonance imaging scan.

10. A model of a patient-specific body part comprising:
a first layer of the patient-specific body part comprising a first material, wherein the first layer has been fabricated according to a first digital model, wherein the first digital model has been created with an algorithm performing a calculation utilizing at least a first input of a medical imaging database comprising data values of medical imaging studies of a patient, wherein the calculation is performed upon a server having a processor and a memory, the memory storing instructions to be executed by the processor, the server coupled to a user terminal and to a communication network;
a second layer of the patient-specific body part comprising a second material, wherein the second layer coats at least a first portion of the first layer of the patient-specific body part;
a third layer of the patient-specific body part comprising a third material, wherein the third layer is affixed to the second layer; and
wherein the second layer forms an intervening layer of the model.

11. The device of claim 10 wherein a characteristic of the second material improves bonding of a third material to the patient-specific body part model.

12. The device of claim 11 wherein the first material comprises one or more of a Stratasys® Vero family material or a Stratasys® Agilus family material and the second material comprises a cyanoacrylate.

13. The device of claim 12 wherein the third material comprises a magnetic material.

14. The device of claim 11 wherein the first material comprises one or more of a Stratasys® Vero family material or a Stratasys® Agilus family material and the second material comprises an epoxy.

15. The device of claim 14 wherein the third material comprises a magnetic material.

16. The device of claim 14 wherein the third material comprises a metallic stud or pin.

17. The device of claim 11 wherein the third material comprises a hook and loop fastener.

18. The device of claim 11 wherein the first material comprises one or more of a Stratasys® Vero family material or a Stratasys® Agilus family material and the second layer is a deposit of the first material, wherein a monomer form of the first material is adhered to the third material before it is affixed to the patient-specific body part.

19. An apparatus for fabricating a model of a patient-specific body part model for a natural body part comprising:
a server having a processor and a memory, wherein the memory stores instructions to be executed by the processor, and wherein the server is coupled to a user terminal and to a communications network, wherein the server stores at least a portion of a medical imaging database comprising data values of medical imaging studies of the patient;
a fabrication apparatus coupled to the server through the communication network, the fabrication apparatus capable of adding multiple materials to a work piece; and
a preform of a first material with a surface upon which an intervening layer and a third material is printed.

20. The apparatus of claim 19 wherein the first material comprises titanium and the third material comprises a medication-infiltrated bioresorbable substrate.
